(19) United States
(12) Patent Application Publication
(10) Pub. No.: US 2012/0059240 A1
(43) Pub. Date: Mar. 8, 2012

(54) METHOD FOR PREPARATION OF MICELLAR HYBRID NANOPARTICLES FOR THERAPEUTIC AND DIAGNOSTIC APPLICATIONS AND COMPOSITIONS THEREOF

(75) Inventors: Michael J. Sailor, La Jolla, CA (US); Sangeeta N. Bhatia, Lexington, MA (US); Ji-Ho Park, Daejeon (KR); Geoffrey A. Von Maltzahn, Boston, MA (US)

(73) Assignees: MASSACHUSETTS INSTITUTE OF TECHNOLOGY, Cambridge, MA (US); THE REGENTS OF THE UNIVERSITY OF CALIFORNIA, Oakland, CA (US)

(21) Appl. No.: 13/219,906

(22) Filed: Aug. 29, 2011

Related U.S. Application Data
(63) Continuation of application No. 13/001,332, now abandoned.
(60) Provisional application No. 61/075,144, filed on Jun. 24, 2008.

(51) Int. Cl. A61B 5/055; A61K 31/704; A61K 49/00; A61K 49/18; A61P 35/00; G01N 33/566; C12Q 1/68; A61N 2/10; A61K 9/14; C12Q 1/02; B82Y 5/00; B82Y 40/00; B82Y 25/00

(52) U.S. Cl. 600/409; 424/490; 514/34; 424/9.1; 424/9.3; 435/29; 435/7.1; 435/6.1; 600/12; 977/773; 977/838; 977/774; 977/915

(57) ABSTRACT

The disclosure provides a long-circulating, micellar hybrid nanoparticles (MHN) that contain MN, QD, and the anti-cancer drug doxorubicin (DOX) within a single polyethylene glycol (PEG)-phospholipid micelle and provide the first examples of simultaneous targeted drug delivery and dual-mode NIR-fluorescent and MR imaging of diseased tissue in vitro and in vivo.

### CROSS REFERENCE TO RELATED APPLICATIONS

This application is continuation of U.S. application Ser. No. 13/001,332, filed Dec. 23, 2010, which is a U.S. National Stage Application filed under 35 U.S.C. §371 and claims priority to International Application No. PCT/US09/48404, filed Jun. 24, 2009, which application claims priority under 35 U.S.C. §119 to U.S. Provisional Application No. 61/075,144, filed Jun. 24, 2008, the disclosures of which are incorporated herein by reference.

### STATEMENT REGARDING FEDERALLY SPONSORED RESEARCH

This invention was made with government support under Grant Nos. R01CA124427-02, CA 119335 and U01 HL 080718 awarded by National Cancer Institute and the National Institutes of Health. The government has certain rights in the invention.

### FIELD OF THE INVENTION

The disclosure relates to micellar-nanoparticle compositions, methods of making and using the same.

### BACKGROUND

Multifunctional nanoparticles have the potential to integrate therapeutic and diagnostic functions into a single nanodevice. While some nanocomposites to date have been used for in vitro magnetic cell separation and in vitro cell targeting, there are limited in vivo studies, particularly for cancer imaging and therapy, due to poor stability or short systemic circulation times generally observed for these more complicated nanostructures.

### SUMMARY

The disclosure provides compositions comprising a micelle hybrid nanostructure having bimodal imaging capacity and drug delivery capacity both in vivo and in vitro. Furthermore, the disclosure provides methods of making and using such micelle hybrid nanostructures. More particularly, the disclosure provides in specific embodiments, micellar hybrid nanostructure that comprise a magnetic nanostructure, at least one quantum dot, QD, and a therapeutic drug (e.g., an anti-cancer drug) within a single PEG-phospholipid micelle. The hydrophobic chains of the PEG-phospholipids interact strongly with hydrophobic character of the magnetic nanostructure (MN) and quantum dot material (QD), providing high dispersibility and stability for in vitro and in vivo applications. The MHN enable dual-mode imaging for cells in vitro and organs in vivo or ex vivo, combining the advantages of optical imaging (for microscopic resolution and in vivo fluorescent imaging) and MRI (for determination of full anatomical distribution in vivo).

The disclosure provides long-circulating, micellar hybrid nanoparticles (MHN) that provides bimodal imaging capabilities. In some embodiments, the MHN comprises a desired cargo agent. The cargo agent can be a therapeutic or diagnostic agent including, but not limited to, anti-cancer agents, polypeptides, RNAi molecules, small molecule drugs and the like. In one embodiment, the MHN comprises a magnetic nanoparticle, quantum dot and comprises the anti-cancer drug doxorubicin (DOX) within a single polyethylene glycol (PEG)-phospholipid micelle. The MHN provides the ability to simultaneously target drug delivery and dual-mode NIR-fluorescent and MR imaging of diseased tissue in vitro and in vivo.

The disclosure further provides methods for the preparation and use of nanoparticles comprising a PEG (poly ethylene glycol)-modified lipid outer layer encapsulating a plurality of nanoparticles or molecular payloads that comprise, for example, iron oxide nanoparticles, quantum dots, and a cargo agent (e.g., such as the anti-cancer agent doxorubicin). Other possibilities for payloads include imaging or contrast agents for Magnetic Resonance Imaging, Positron Emission Tomography, X-ray imaging, Fluorescence imaging, or other medical imaging technologies, therapeutic agents (e.g., polypeptide, small molecule drugs, RNAi), vaccines, and adjuvants.

The disclosure provides micelle compositions encapsulating a plurality of different nanostructures at least two of the plurality of nanostructures having different excitation/emission spectrums or detectable signals. In one embodiment, the at least one nanostructure comprises a magnetic material. In another embodiment, the at least one nanostructure comprises a quantum dot. In yet a further embodiment, the plurality of nanostructures comprise at least one quantum dot and at least one magnetic nanostructure. The micelle may further encapsulate a therapeutic drug. In some embodiments, the therapeutic drug is an anticancer drug such as a member selected from the group consisting of methotrexate (Abitrexate®), fluorouracil (Adrucil®), hydroxyurea (Hydrea®), mercaptopurine (Purinethol®), cisplatin (Platinol®), daunorubicin (Cerubidine®), doxorubicin (Adriamycin®), etoposide (VePesid®), Vinblastine (Velban®), Vincristine (Oncovin®) and Pacitaxel (Taxol®). In some embodiments, the compositions (e.g., the micelle, PEG-lipid, nanoparticle) may be conjugated to a targeting moiety such as a receptor, a receptor ligand or an antibody. In yet other embodiments the lipid of the micelle are pegylated.

The disclosure also provides micelle compositions encapsulating a plurality of different nanostructures at least two of the plurality of nanostructure comprising different materials. In one embodiment, the at least one nanostructure comprises a magnetic material. In another embodiment, the at least one nanostructure comprises a quantum dot. In yet a further embodiment, the plurality of nanostructures comprise at least one quantum dot and at least one magnetic nanostructure.

The micelle may further encapsulate a therapeutic drug. In some embodiments, the therapeutic drug is an anticancer drug such as a member selected from the group consisting of methotrexate (Abitrexate®), fluorouracil (Adrucil®), hydroxyurea (Hydrea®), mercaptopurine (Purinethol®), cisplatin (Platinol®), daunorubicin (Cerubidine®), doxorubicin (Adriamycin®), etoposide (VePesid®), Vinblastine (Velban®), Vincristine (Oncovin®) and Pacitaxel (Taxol®). In some embodiments, the compositions (e.g., the micelle, PEG-lipid, nanoparticle) may be conjugated to a targeting moiety such as a receptor, a receptor ligand or an antibody. In yet other embodiments the lipid of the micelle are pegylated.

The disclosure also provides a method of making a pegylated-micelle-nanostructure composition comprising: evaporating a mixture comprising pegylated lipids, at least one nanostructure, at least one quantum dot and an organic solvent to obtain a dry mixture; hydrating the dry mixture in a hydrating medium to obtain a pegylated-micelle-nanostructure composition, wherein the nanostructure and quantum dot are encapsulated within the micelle. In some embodiments, the method further comprises adding a drug to either of the organic solvent or the hydrating medium. In yet other embodiments, the pegylated lipid is conjugated to a targeting moiety. In certain embodiments, the drug is an anticancer drug such as one selected from the group consisting of methotrexate (Abitrexate®), fluorouracil (Adrucil®), hydroxyurea (Hydrea®), mercaptopurine (Purinethol®), cisplatin (Platinol®), daunorubicin (Cerubidine®), doxorubicin (Adriamycin®), etoposide (VePesid®), Vinblastine (Velban®), Vincristine (Oncovin®) and Pacitaxel (Taxol®).

The disclosure further provides pharmaceutical compositions comprising the micelle-nanostructure (MHN) described herein.

The disclosure also provides a method of treating or diagnosing a disease or disorder in a subject comprising administering a composition comprising an MHN or an MHN to a subject and contacting the subject with a device that can detect the magnetic rotation of a nanostructure.

The disclosure also provides a method of treating or diagnosing a disease or disorder in a subject comprising administering a composition comprising an MHN or an MHN to a subject and contacting the subject with a device that excites the nanostructure to induce vibration or thermal energy and the site of the nanostructure.

The micellar nanoparticles exhibit substantial in vivo circulation times and significant tumor targeting when coated with tumor-homing peptides or binding agents. In one embodiment, the disclosure provides methods for chemically attaching polyethylene glycol to the lipid elements that constitute the micellar coating. The resulting micelles exhibit low permeability, allowing them to contain a diverse payload for periods of time sufficient to allow them to circulate in the body and locate a desired tissue before releasing the cargo/payload.

### BRIEF DESCRIPTION OF THE FIGURES

FIG. 1A-F shows transmission electron microscope images of (a) micellar hybrid nanoparticles (MHN) with a mass ratio of 1 [magnetic nanoparticles (MN)] to 5 [quantum dots (QD)] (inset: TEM image of MHN with negative staining by 1.3% phosphotungstic acid), (b-d) magnified images of MHN with a mass ratio of (b) MHN1 with 1(MN):1(QD), (c) MHN3 with 1(MN):3(QD), and (d) MHN5 with 1(MN):5(QD). (e) micellar magnetic nanoparticles, (f) micellar quantum dots (emission λₘₐₓ=705 nm). Note that micellar coating layer of MHN was observed with brighter color in the negative stained TEM image [inset in (a)]. Scale bar in (a) is 100 nm, scale bar in (b) is 20 nm for [inset in (a) and (b-d)], and scale bar in (e) is 20 nm for (e and f). In these formulations the QD are somewhat elongated and the MN are spherical.

FIG. 2A-C shows: (a) Photoluminescence spectra of micellar quantum dots (MQD, emission λₘₐₓ=705 nm), micellar magnetic nanoparticles (MMN) and micellar hybrid nanoparticles (MHN) with different compositions of MN and QD. The particle samples were excited with 450 nm light. The spectra are normalized by total mass of each particle type. (b) Multimodal imaging of MMN and MHN as a function of iron concentration in MRI (upper panel, T₂-weighted mode) and NIR fluorescence (lower, in the Cy5.5 fluorescence channel λₑₓ=680 nm, λ_{obs}=720 nm). (c) Relaxivity R₂ values of MMN and MHN in the T₂-weighted MR images.

FIG. 3A-C shows: (a) Intracellular delivery of F3-conjugated micellar hybrid nanoparticles (F3-MHN) into MDA-MB-435 human carcinoma cells. In both panels the F3-MHN or the MHN control particles appear red in the images. 2 h after incubation with the cells, the F3-MHN particles are strongly associated with the cells, while the control nanoparticles (MHN) without the F3 species do not penetrate. (b) Multimodal images (NIR fluorescence in Cy5.5 channel and MRI) of the cells in (a) compared with PBS control and with untreated cells. (c) Targeted drug delivery of doxorubicin (DOX)-incorporated F3-MHN into MDA-MB-435 human carcinoma cells. The DOX-loaded F3-MHN were incubated with the cells for 2 h. Arrowheads indicate co-localization of DOX and MHN. The inset shows co-localization of DOX and endosome marker 30 min after incubation with DOX-loaded F3-MHN. Nuclei were stained with DAPI.

FIG. 4A-B shows: (a) NIR fluorescence images of in vivo passive accumulation of micellar hybrid nanoparticles containing the QD emitting at 800 nm [MHN(800)] in a mouse bearing MDA-MB-435 tumors. The mouse was imaged pre-injection and 20 h post-injection (injection dose: 10 mg/Kg). (b) Multimodal imaging of ex vivo tumor harvested from the mouse in (a) in MRI and NIR fluorescence (control: PBS-injected tumor). NIRFI indicates near-infrared fluorescence image and MRI(T₂) indicates T₂ values in T₂-weighted mode MRI.

FIG. 5 shows SQUID magnetization curves for MMN and MHN3 samples. The magnetization values are normalized by the total mass of particles in each sample.

FIG. 6 shows fluorescence spectra of micellar hybrid nanoparticles (MHN) and doxorubicin-loaded MHN (DOX-MHN), obtained using 480 nm excitation. The weak fluorescence observed in the wavelength range 540-630 nm for the DOX-MHN sample is attributed to intrinsic fluorescence from DOX.

FIG. 7 shows targeted intracellular drug delivery of doxorubicin (DOX)-incorporated F3-MHN (DOX-MHN-F3) into MDA-MB-435 human carcinoma cells at multiple time points. The left and middle panels are for DOX-incorporated F3-MHN. Nuclei are stained with DAPI. The right panels are for free DOX that is physically mixed with F3-MHN (not incorporated into the MHN).

FIG. 8 shows cytotoxicity of various formulations of MHN by MTT assay. MDA-MB-435 human carcinoma cells are treated with free DOX, MHN, DOX-incorporated MHN (DOX-MHN), and DOX-incorporated F3-MHN (DOX-MHN-F3) for 4 h. The amounts of DOX and MHN used here are equivalent for all formulations (˜0.093 mg of DOX per mg of MHN).

FIG. 9A-B shows (a) TEM image of MHN composed of MN and QD emitting at 800 nm [MHN(800)]. Scale bar is 50 nm. (b) Photoluminescent spectra of MMN, MQD(800), and MHN(800), obtained with 450 nm excitation.

FIG. 10A-C shows (a) TEM image of MHN recovered from the blood circulation in mouse 2 h after intravenous injection (negative staining by 1.3% phosphotungstic acid). Scare bar is 50 nm. (b) Biodistribution of MHN(800) 20 h after injection with a dose of 10 mg/kg. The organs were imaged in the Cy7 channel using a NIR fluorescence imaging (NIRFI) system. (c) ex vivo NIRF and MR images of tumors harvested from mice 20 h after injection of either MHN(800) (green in NIRF images), or MHN(705) (red in NIRF images). MHN(800) or MHN(705) doses for the injections were 10 mg/kg. The control corresponds to tumors injected with equivalent volumes of phosphate-buffered saline (PBS). MR images obtained in T₂-weighted mode; the color map for the T₂ values is indicated at the far right of the Figure.

FIG. 11 shows a synthetic procedure used to prepare micellar hybrid nanoparticles that encapsulate magnetic nanoparticles and quantum dots within a single PEG-phospholipid micelle.

### DETAILED DESCRIPTION

As used herein and in the appended claims, the singular forms “a,” “and,” and “the” include plural referents unless the context clearly dictates otherwise. Thus, for example, reference to “a nanoparticle” includes a plurality of such nanoparticle and reference to “the cell” includes reference to one or more cells known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

Micellar preparations of hydrophobic drugs and nanoparticles using diblock polymers hold great potential for biomedical applications. Such micellar coatings can display excellent stability, reducing the cytotoxicity of the hydrophobic drug or nanoparticle contents. Previous in vitro studies have demonstrated that drug molecules and magnetic nanoparticles (MN) can be incorporated within a micelle, allowing the corroboration of drug delivery by MRI. Furthermore, micellar preparations containing single-component nanomaterials such as QD and carbon nanotubes have been shown to be sufficiently stable for in vivo applications.

Targeted delivery of therapeutics and diagnostics to a tissue or cell both in vivo and in vitro have been attempted and to some degree have been successful. Such techniques have used ligand binding domains associated with a particular therapeutic or diagnostic. However, the half-life of such molecules differs dramatically depending upon the composition. For example, the ability to sufficiently deliver the therapeutic or diagnostic is dependent upon additional factor such as dosing and the use of secondary active agents to modulate immune functionality. The compositions of the disclosure improve the circulating times of therapeutic compositions thus improving their delivery and efficacy.

The disclosure provides micellar compositions for imaging and therapeutic delivery comprising at least two compositionally different or geometrically different nanostructures. The disclosure provides compositions comprising (1) at least two compositionally or geometrically different nanostructures, and (2) a phospholipid micelle. In some embodiments, the phospholipids are pegylated. In one embodiment, at least one of the at least two nanostructures is a quantum dot (QD). In one embodiment, the composition comprises a nanostructure and at least one quantum dot encapsulated within a PEG-phospholipid micelle. The composition may further include an additional active agent as a cargo/payload within or associated with the micelle composition (e.g., such as an anticancer agent). In another embodiment, the disclosure provides a nanostructure linked or associated with the micellar structure and an active agent encapsulated within the micelle. In yet a further embodiment, the nanostructure or micelle may be further functionalized to include a targeting moiety.

By “encapsulation”, it is meant stable association with the a micelle structure. Thus, it is not necessary for the micelle to surround the nanostructure(s), agent or agents so long as the nanostructure(s), agent or agents is/are stably associated with the micelle when administered in vivo. Thus, “stably associated with” and “encapsulated in” or “encapsulated with” or “co-encapsulated in or with” are intended to be synonymous terms. They are used interchangeably in this specification. The stable association may be effected by a variety of means, including covalent bonding, noncovalent bonding, and trapping in the interior of the micelle and the like. The association must be sufficiently stable so that the agents or nanostructure remain associated with the delivery vehicle at a non-antagonistic until it is delivered to a target site or for a desired period of time.

The MHN composition of the disclosure comprise a lipid in the form of a liposome, lipid micelle, lipoprotein micelle and the like. In certain embodiments, a cholesterol-free liposomes containing PG or PI is used to prevent aggregation thereby increasing the blood residence time of the carrier.

Micelles are self-assembling particles composed of amphipathic lipids or polymeric components that are typically utilized for the delivery various active agents. Various means for the preparation of micellar delivery vehicles are available and may be carried out with ease by one skilled in the art. The term “micelle” or its cognates can be used to describe a lipid monolayer, which is distinguished from a liposome which is a lipid bilayer. Phospholipids are molecules that contain long hydrophobic tail at one end, and a polar head at the other end. For instance, lipid micelles may be prepared as described in Perkins, et al., Int. J. Pharm. (2000) 200(1):27-39 (incorporated herein by reference). Lipoprotein micelles can be prepared from natural or artificial lipoproteins including low and high-density lipoproteins and chylomicrons. Lipid-stabilized emulsions are micelles prepared such that they comprise an oil filled core stabilized by an emulsifying component such as a monolayer or bilayer of lipids. The core may comprise fatty acid esters such as triacylglycerol (corn oil). The monolayer or bilayer may comprise a hydrophilic polymer lipid conjugate such as DSPE-PEG. These delivery vehicles may be prepared by homogenization of the oil in the presence of the polymer lipid conjugate. Agents that are incorporated into lipid-stabilized emulsions are generally poorly water-soluble. Synthetic polymer analogues that display properties similar to lipoproteins such as micelles of stearic acid esters or poly(ethylene oxide) block-poly(hydroxyethyl-L-aspartamide) and poly(ethylene oxide)-block-poly(hydroxyhexyl-L-aspartamide) may also be used in the practice of the embodiments of the disclosure (Lavasanifar, et al., J. Biomed. Mater. Res. (2000) 52:831-835).

Liposomes and micelles are used as carriers for drugs and antigens because they can serve several different purposes (Storm & Crommelin, Pharmaceutical Science & Technology Today, 1, 19-31 1998). Liposome and micelle encapsulated drugs are inaccessible to metabolizing enzymes. Conversely, body components (such as erythrocytes or tissues at the injection site) are not directly exposed to the full dose of the drug. Liposomes and micelles possessing a direction potential, that means, targeting options change the distribution of the drug over the body. Cells use endocytosis or phagocytosis mechanism to take up liposomes and micelles into the cytosol. Furthermore liposomes and micelles can protect a drug against degradation (e.g. metabolic degradation).

Therapeutic agents can be used with or encapsulated within the micelle compositions of the disclosure. A “therapeutic agent” is a compound that alone, or in combination with other compounds, has a desirable effect on a subject affected by an unwanted condition or disease.

In one embodiment, the therapeutic agent is an anticancer agent. An anticancer agent can be encapsulated within the micelle. Suitable anticancer drugs can be selected from the group consisting of methotrexate (Abitrexate®), fluorouracil (Adrucil®), hydroxyurea (Hydrea®), mercaptopurine (Purinethol®), cisplatin (Platinol®), daunorubicin (Cerubidine®), doxorubicin (Adriamycin®), etoposide (VePesid®), Vinblastine (Velban®), Vincristine (Oncovin®) and Pacitaxel (Taxol®).

Other therapeutic agents include signal transduction inhibitors, which interfere with or prevents signals that cause cancer cells to grow or divide; cytotoxic agents; cell cycle inhibitors or cell cycle control inhibitors, which interfere with the progress of a cell through its normal cell cycle, the life span of a cell, from the mitosis that gives it origin to the events following mitosis that divides it into daughter cells; checkpoint inhibitors, which interfere with the normal function of cell cycle checkpoints, e.g., the S/G2 checkpoint, G2/M checkpoint and G1/S checkpoint; topoisomerase inhibitors, such as camptothecins, which interfere with topoisomerase I or II activity, enzymes necessary for DNA replication and transcription; receptor tyrosine kinase inhibitors, which interfere with the activity of growth factor receptors that possess tyrosine kinase activity; apoptosis inducing agents, which promote programmed cell death; antimetabolites, such as Gemcitabine or Hydroxyurea, which closely resemble an essential metabolite and therefore interfere with physiological reactions involving it; telomerase inhibitors, which interfere with the activity of a telomerase, an enzyme that extends telomere length and extends the lifetime of the cell and its replicative capacity and the like.

All synergistic or additive combinations of agents are within the scope of the disclosure. Typically, for the treatment of a neoplasm, combinations that inhibit more than one mechanism that leads to uncontrolled cell proliferation are chosen for use in accordance with this disclosure. For example, the disclosure includes selecting combinations that effect specific points within the cell cycle thereby resulting in non-antagonistic effects. For instance, drugs that cause DNA damage can be paired with those that inhibit DNA repair, such as anti-metabolites.

Specific agents that may be used in combination include cisplatin (or carboplatin) and 5-FU (or FUDR), cisplatin (or carboplatin) and irinotecan, irinotecan and 5-FU (or FUDR), vinorelbine and cisplatin (or carboplatin), methotrexate and 5-FU (or FUDR), idarubicin and araC, cisplatin (or carboplatin) and taxol, cisplatin (or carboplatin) and etoposide, cisplatin (or carboplatin) and topotecan, cisplatin (or carboplatin) and daunorubicin, cisplatin (or carboplatin) and doxorubicin, cisplatin (or carboplatin) and gemcitabine, oxaliplatin and 5-FU (or FUDR), gemcitabine and 5-FU (or FUDR), adriamycin and vinorelbine, taxol and doxorubicin, flavopuridol and doxorubicin, UCN01 and doxorubicin, bleomycin and trichlorperazine, vinorelbine and edelfosine, vinorelbine and sphingosine (and sphingosine analogues), vinorelbine and phosphatidylserine, vinorelbine and camptothecin, cisplatin (or carboplatin) and sphingosine (and sphingosine analogues), sphingosine (and sphingosine analogues) and daunorubicin and sphingosine (and sphingosine analogues) and doxorubicin.

Some lipids are “therapeutic lipids” that are able to exert therapeutic effects such as inducing apoptosis. Included in this definition are lipids such as ether lipids, phosphatidic acid, phosphonates, ceramide and ceramide analogues, dihydroxyceramide, phytoceramide, sphingosine, sphingosine analogues, sphingomyelin, serine-containing lipids and sphinganine The term “serine-containing phospholipid” or “serine-containing lipid” as defined herein is a phospholipid in which the polar head group comprises a phosphate group covalently joined at one end to a serine and at the other end to a three-carbon backbone connected to a hydrophobic portion through an ether, ester or amide linkage. Included in this class are the phospholipids such as phosphatidylserine (PS) that have two hydrocarbon chains in the hydrophobic portion that are between 5-23 carbon atoms in length and have varying degrees of saturation. The term hydrophobic portion with reference to a serine-containing phospholipid or serine-containing lipid refers to apolar groups such as long saturated or unsaturated aliphatic hydrocarbon chains, optionally substituted by one or more aromatic, alicyclic or heterocyclic group(s).

“Pegylated lipid” is used herein to indicate a lipid which is conjugated to a polyethylene glycol (PEG) moiety. In one embodiment a PEG-phospholipid is used for the formation of a micelle structure. A PEG-phospholipid can comprise a combination of dipalmitoyl phosphatidyl choline, dipalmitoyl phosphatidyl glycerol and pegylated distearoyl phosphatidyl ethanolamine. In particular embodiments, where the lipid species comprises a pegylated lipid, the total content of pegylated lipid, as a percentage of total lipid content, will be in the range of 1% to approximately 20% or more. In other embodiments the range of pegylated lipid will be approximately 1-10%, approximately 1-6%, approximately 1-5%, approximately 1-4% or approximately 1-3%. In certain embodiments, the total content of pegylated lipid will be approximately 2.5%, approximately 4%, approximately 5%, approximately 10%, approximately 15% or approximately 20%. A complex may contain both pegylated and non-pegylated lipid of a particular type, for example, pegylated and non-pegylated DSPE. In certain embodiments, the total pegylated lipid content is no more than approximately 10%.

“Polyethylene glycol” and “PEG” refer to compounds of the general formula H(OCH₂CH₂)ₙOH, wherein n may be any integer greater than 1. Typical PEG formulations have an average molecular weight of about 750-20,000. As used herein, “PEG” and “polyethylene glycol” are meant to encompass PEG compositions which may optionally include one or more functional groups (such as, for example, methoxy, biotin, succinyl, nickel or conjugating PEG to another moiety, such as a lipid or a targeting factor.

“Targeting factor-pegylated lipid conjugate” is used herein to indicate a targeting factor which has been conjugated to a pegylated lipid. The targeting factor may be conjugated, for example, to the PEG moiety of the pegylated lipid.

As mentioned above, liposomes and micelles are often rapidly cleared or taken up by various organs of the body (e.g., the liver, spleen, kidneys, etc.). Pegylation is an alternative method to overcome these deficiencies. First, pegylation maintains drug levels within the therapeutic window for longer time periods and provides the drug as a long-circulating moiety that gradually degrades into smaller, more active, and/or easier to clear fragments. Second, it enables long-circulating drug-containing micro particulates or large macromolecules to slowly accumulate in pathological sites with affected vasculature or receptor expression and improves or enhances drug delivery in those areas. Third, it can help to achieve a better targeting effect for those targeted drugs and drug carriers which are supposed to reach pathological areas with diminished blood flow or with a low concentration of a target antigen. The benefits of pegylation typically result in an increased stability (temperature, pH, solvent, etc.), a significantly reduced immunogenicity and antigenicity, a resistance to proteases, a maintenance of catalytic activity, and improvements in solubility, among other features, and an increased liquid stability of the product and reduced agitation-induced aggregation.

Poly (ethylene glycol)-linked lipids (PEG-lipid) or gangliosides containing doxorubicin are useful to treat cell proliferative disorders. The presence of MPEG-derivatized (pegylated) lipids effectively furnishes a steric barrier against interactions with plasma proteins and cell surface receptors that are responsible for the rapid intravascular destabilization/rupture. As a result, pegylated lipids have a prolonged circulation half-life, and the pharmacokinetics of any encapsulated agent are altered to conform to those of the liposomal carrier rather than those of the entrapped drug (Stewart et al., J. Clin. Oncol. 16, 683-691, 1998). Because the mechanism of tumor localization of pegylated material is by means of extravasation through leaky blood vessels in the tumor (Northfelt et al., J. Clin. Oncol. 16, 2445-2451, 1998; Muggia et al., J. Clin. Oncol. 15, 987-993, 1997), prolonged circulation is likely to favor accumulation in the tumor by increasing the total number of passes made by the pegylated liposomes through the tumor vasculature.

Any of a various number of nanostructure of metallic, metal alloy, layered metallic materials or biocompatible materials can be used in the methods and compositions of the disclosure.

Metals, alloys and materials useful for the formation of a nanostructure of the disclosure can be obtained based upon a functional layer or thermal bias layer. The material is selected from the group of noble metal and transition metal, including but not limited to Ag, Au, Cu, Al, Fe, Co, Ni, Ru, Rh, Pd, and Pt. A further surface functional layer can be added or formed in combination with the noble or transition metal core material. Such functional layers can include, but are not limited to, Ag oxide, Au oxide, SiO₂, Al₂O₃, Si₃N₄, Ta₂O₅, TiO₂, ZnO, ZrO₂HfO₂, Y₂O₃, Tin oxide, antimony oxide, and other oxides; Ag doped with chlorine or chloride, Au doped chlorine or chloride, Ethylene and Chlorotrifluoroethylene (ECTFE), Poly(ethylene-co-butyl acrylate-co-carbon monoxide) (PEBA), Poly(allylamine hydrochloride) (PAH), Polystyrene sulfonate (PSS), Polytetrafluoroethylene (PTFE), Polyvinyl alcohol (PVA), Polyvinyl chloride (PVC), Polyvinyldene fluoride (PVDF), Polyvinylprorolidone (PVP), and other polymers; stacked multiple layers at least two layers including above listed metal layers and non-metal layers, and the like. A typical material is a metal such as Au, Ag, Ti, Ni, Cr, Pt, Ru, Ni—Cr alloy, NiCrN, Pt—Rh alloy, Cu—Au—Co alloy, Ir—Rh alloy or/and W—Re alloy. The material used should be biocompatible.

The nanostructure of the disclosure can comprise any number of different or combinations of paramagnetic metals in order to form a contrast agent for use in MRI. Typically such paramagnetic metal ions have atomic numbers 21-29, 42, 44, or 57-83. This includes ions of the transition metal or lanthanide series which have one, and more, typically, five or more, unpaired electrons and a magnetic moment of at least 1.7 Bohr magneton. Exemplary paramagnetic metals include, but are not limited to, chromium (III), manganese (II), manganese (III), iron (II), iron (III), cobalt (II), nickel (II), copper (II), praseodymium (III), neodymium (III), samarium (III), gadolinium (III), terbium (III), dysprosium (III), holmium (III), erbium (III), europium (III) and ytterbium (III).

Gd(III) is particularly useful for MRI due to its high relaxivity and low toxicity, and the availability of only one biologically accessible oxidation state. Gd(III) chelates have been used for clinical and radiologic MR applications since 1988, and approximately 30% of MR exams currently employ a gadolinium-based contrast agent.

One skilled in the art will be able to select a metal according to the intended use, dose required to detect a target tissue/cell as well as considering other factors such as toxicity of the metal to the subject. See, Tweedle et al., Magnetic Resonance Imaging (2nd ed.), vol. 1, Partain et al., eds. (W. B. Saunders Co. 1988), pp. 796-7. Generally, the desired dose for an individual metal will be proportional to its relaxivity, modified by the biodistribution, pharmacokinetics and metabolism of the metal. The trivalent cation, Gd³⁺ is particularly useful for MRI contrast agents, due to its high relaxivity and low toxicity, with the further advantage that it exists in only one biologically accessible oxidation state, which minimizes undesired metabolization of the metal by a patient.

The geometry or structure of the nanostructure can incorporate the functional capabilities of nanotip, nanosphere, and nanoring geometries. Other geometries can include spherical, circular, triangle, quasi-triangle, square, rectangular, hexagonal, oval, elliptical, rectangular with semi-circles or triangles and the like. The nanostructures of the materials and geometries ideally have an absorbance or excitation wavelength in the near infrared range. Selection of suitable materials and geometries are known in the art. Excitation at longer wavelengths provides deeper penetration into tissue with minimal photothermal damage, and excitation of the nanostructure does not cause fluorescence of other biomolecules.

Various nanostructure geometries are capable near-infrared (NIR) excitation. For example, nanopins, crescents, bowls, hollow spheres and the like (see, e.g., International Application Publ. No. WO/2006/099494, the disclosure of which is incorporated herein) have a higher local field-enhancement factor in the near-infrared wavelength region due to the simultaneous incorporation of SERS hot spots including sharp nanotip and nanoring geometries, leading to the strong hybrid resonance modes from nanocavity resonance modes and tip-tip intercoupling modes.

One of skill in the art will recognize that the size, shape, and thickness or, where multi-layers are present, layer thickness can all be individually controlled by modifying the size of a sacrificial nanostructure template, the deposition angle, the deposited layer thickness, and the material of each layer. Since the plasmon-resonance wavelength of the metallic nanostructures is dependent on these parameters, the optical properties of the nanostructure are tunable during fabrication.

The compositions of the disclosure also include quantum dots. Quantum dots (QDs), are a class of nanoparticles that have been the focus of research and have demonstrated remarkable potential for commercial applications. QDs may exhibit semiconducting, fluorescence, or emissive characteristics.

The disclosure takes advantage of the emission characteristics of QDs for detection of a composition of the disclosure. Typically a QD is a semiconductor nanocrystal whose radius is smaller than the bulk excitation Bohr radius. QDs may be formed from inorganic, crystalline semiconductive materials and, among other things, have unique photophysical, photochemical, and nonlinear optical properties arising from quantum size effects. Typically, QDs are composed of inorganic matter and therefore, they are normally insoluble in water.

QDs may be formed from an inner core of one or more first semiconductor materials that optionally may be contained within an overcoating or “shell” of a second semiconductor material. A QD core surrounded by a semiconductor shell is referred to as a “core/shell” QD. In certain embodiments, the optional surrounding shell material will have a bandgap energy that is larger than the bandgap energy of the core material and may be chosen to have an atomic spacing close to that of the core substrate. Suitable semiconductor materials for the core and/or the optional shell include, but are not limited to, the following: materials comprised of a first element selected from Groups 2 and 12 of the Periodic Table of the Elements and a second element selected from Group 16 (e.g., ZnS, ZnSe, ZnTe, CDs, CdSe, CdTe, HgS, HgSe, HgTe, MgS, MgSe, MgTe, CaS, CaSe, CaTe, SrS, SrSe, SrTe, BaS, BaSe, BaTe, and the like); materials comprised of a first element selected from Group 13 of the Periodic Table of the Elements and a second element selected from Group 15 (e.g., GaN, GaP, GaAs, GaSb, InN, InP, InAs, InSb, and the like); materials comprised of a Group 14 element (Ge, Si, and the like); materials such as PbS, PbSe and the like; and alloys and mixtures thereof. QDs may be made using techniques known in the art. See, e.g., U.S. Pat. Nos. 6,048,616; 5,990,479; 5,690,807; 5,505,928; and 5,262,357.

The disclosure provides for methods of detecting, sensing, imaging or treating cells or a tissue in vivo, or in vitro by contacting the cell or tissue with an effective amount of a nanostructure (e.g., a nanoparticle) encapsulated in a micelle formulation. In some embodiments, the micelle-nanostructure, may further comprise a targeting moiety such as a receptor, receptor ligand or antibody. In further embodiments, the micelle-nanostructure further comprises at least one quantum-dot in addition to a metallic or magnetic nanostructure.

In a specific embodiment, the composition of the disclosure comprises a central metallic/magnetic nanostructure core, surrounded by at least two quantum dots and encapsulated within a PEG-lipid micelle. In yet a further embodiment, the composition further comprises a therapeutic agent encapsulated within the micelle. As described above, the therapeutic agent can be a small molecule drug, an anti-cancer agent, and the like.

The disclosure provides a PEG-lipid micelle comprising at the center a cluster of at least one (e.g., 2, 3, 4, 5, 6, 7, 8, 9 or more) quantum dots and at least one magnetic nanostructure. The micellar hybrid nanoparticles (MHNs) of the disclosure comprise clusters of both magnetic nanostructures (MN) and quantum dots (QD) within a micellar coating. In one embodiment, the MHNs comprise a hydrodynamic size of about 10-100 nm (e.g., 20-90 nm, 30-80 nm, 40-70 nm, 60-70 nm or any range there between. The MN/QD ratio within the micelles can be adjusted by changing the mass ratio of MNs to QDs during synthesis.

The disclosure further comprises PEG-micelle encapsulated hybrid materials (e.g., quantum dots and magnetic particles, quantum dots and a drug, imaging agent or other factor or any combination thereof). For example, the disclosure provides hybrid encapsulated materials comprising a quantum dot and a magnetic particle. In one aspect, the disclosure provides a PEG-micelle comprising a nanostructure and an anticancer agent.

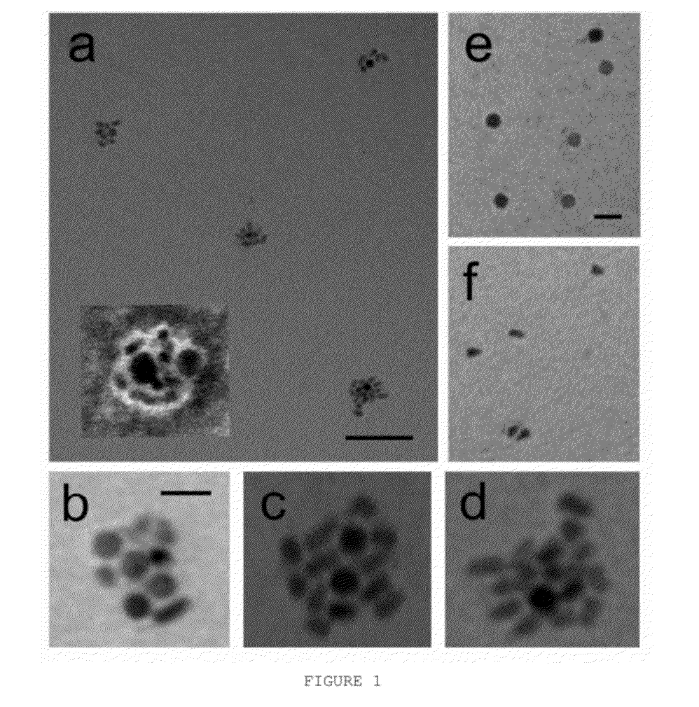

In a specific embodiment, the disclosure provides spherical oleic-acid coated MN with a size of 11 nm and elongated TOP-coated QD with a longitudinal size of 1012 nm and NIR emission wavelength were encapsulated simultaneously within a micelle composed of PEG-phospholipid, (FIG. 11). The micellar MN (MMN), micellar QD (MQD) and empty micelles produced during MHN synthesis were removed by magnetic separation and centrifugation. Transmission electron microscope images and dynamic light scattering measurements reveal that the MHN consist of clusters of both MN and QD within micellar coating with a hydrodynamic size of 60-70 nm (FIG. 1a-d). The MN:QD ratio within the individual micelles can be adjusted by changing the mass ratio of MN to QD during the synthesis. By contrast, MMN or MQD prepared by encapsulating either MN or QD alone with PEG-phospholipids appear to be either individually encapsulated or encapsulated as dimers, respectively (FIGS. 1e and 1f). When relatively concentrated solutions (>2 mg/mL) of MN and QD are added to the PEG-phospholipid solution during micelle formation, aggregates rather than isolated nanoparticles are observed to form. Preparations of 1 mg/mL MHN are stable in either deionized water or in phosphate buffered saline (PBS) solutions, with no observable aggregation or dissociation for at least 1 month. Unlike dispersed arrangements of MN and QD in previous hybrid systems, the MN and QD in the MHN are closely packed within a single micelle, similar to the clustering of MN that have been observed inside poly(caprolactone)-PEG copolymer systems.

As described above, any number of different nanostructure compositions and geometries can be used in the methods and compositions of the disclosure. The nanostructures are typically between about 2-50 nm (e.g., 4-25, 7-14 nm and any range there between) and can be encapsulated within PEG phospholipid micelles. For example, in a solution of chloroform, the mPEG 750 phospholipid forms micelles with the non-polar hydrophobic chains at the center of the micelle, and the polar head on the micelle surface.

To create water-soluble nanoparticle that can be conjugated to antibodies or other molecular targets thiol-functionalized phospholipids (e.g., PTE, phosphatidylthioethanol) can be used. The ratio of functionalized phospholipid and PEG can be modified as desired. For example, the phospholipid micelle comprises from about 0.1% to about 10% functionalized phospholipids. In yet another embodiment, the phospholipid micelle comprises about 1% functionalized phospholipids.

The disclosure also provides methods for synthesis and conjugation phospholipids, PEG, or the nanostructure to other agents of interest. For example, the disclosure includes methods of conjugating iron oxide nanoparticles to antibodies for targeting specific cells using fluorescence and MR imaging techniques.

The nanoparticles can be conjugated to antibodies via a heterobifunctional crosslinker molecule. The pyridyl disulfide end will react with the free thiol groups at the surface of the phospholipids micelle to form a nanoparticle-Antibody conjugate and then the NHS ester will react with amino acids on the antibody surface, such as lysine or arginine.

The disclosure provides methods for imaging specific cells in a body of a subject with an MRI scanner. The disclosure also provides a method for detecting a cell of interest in a subject, the method comprising administering to the subject an effective amount of an MHN (e.g., PEG-micelle-nanoparticle conjugate). In one embodiment, the MHNs of the disclosure comprise an antibody linked to the micellar composition, wherein the antibody specifically binds to an antigen. In certain embodiments of the methods of the disclosure, the nanoparticle is detected by magnetic resonance imaging.

The disclosure can also utilize a functionalized phospholipid to attach antibodies via a crosslinker. In one embodiment of the disclosure, the functionalized phospholipids comprise thiol-functionalized phospholipids, amine functionalized phospholipids, or any combination thereof. In another embodiment, the amine-functionalized phospholipids comprise DSPE-PEG(2000)Carboxylic Acid, DSPE-PEG(2000)Maleimide, DSPE-PEG(2000)PDP, DSPE-PEG(2000)Amine, DSPE-PEG(2000)Biotin, or any combination thereof. In yet another embodiment, the thiol-functionalized phospholipids comprise phophatidylthioethanol (PTE).

The composition of the disclosure can be administered to a subject in the form of an injectable composition. The method of administering the composition (e.g., MRI contrast agent or a therapeutic agent) is typically parenterally, meaning intravenous, intra-arterial, intra-thecal, or interstitial.

For MRI measurements, a subject will receive a dosage of a composition comprising an MHN of the disclosure sufficient to provide a measurable signal at the target. After injection of a composition of the disclosure the subject is scanned in an imaging machine (e.g., an MRI) to determine the location of any sites containing a composition of the disclosure. In a therapeutic setting, upon target localization, the composition may be “activated” by resonance energy or the like to result in a localized treatment.

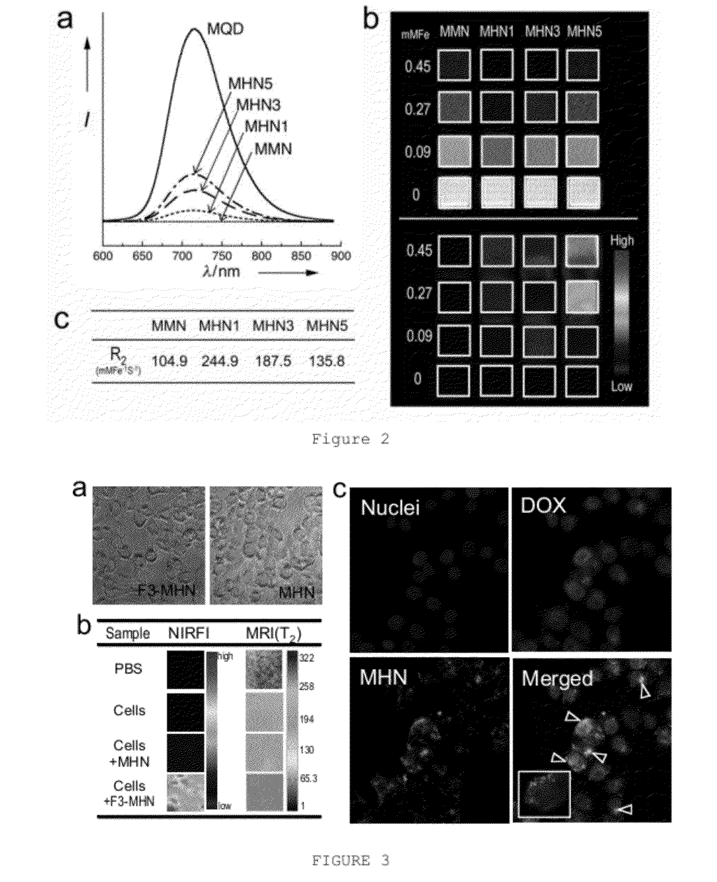

The disclosure demonstrates that the compositions of the disclosure can be remotely imaged by both fluorescence and MRI. For example, MHN preparations, MHN fluorescence was measured with blue (450 nm) and NIR (680 nm) excitation (FIG. 2). At both measurements, as the ratio of MN to QD within a micelle increases, the intensity of fluorescence from the MHN assembly decreases with no significant spectral shift or line broadening of the emission spectrum. The loss of fluorescence intensity can be attributed to decreased number of QD per a micelle and optical absorption by the MN. Additionally, the proximity of MN and other QD in the MHN can cause some fluorescence quenching through non-radiative energy or charge transfer. Despite the quenching, the fluorescence is strong enough to allow detection of MHN at sub-nanomolar QD concentrations. These inorganic QD-containing hybrid systems can be excited and observed in the NIR spectral region with high photostability, providing significant advantages over the MN labeled with organic fluorophores.

The MHN materials can also be effectively imaged by MRI. The MR characteristics of MHN with varying MN:QD ratios were compared to MMN (FIGS. 2b and 2c). The T₂-weighted images of MHN1 and MHN3, composed of MN clusters, reveal significantly larger MR contrast compared to MMN (T₂ relaxation rates R₂=244.9, 187.5, and 104.9 mMFe⁻¹S⁻¹, respectively). The increased T₂ relaxivity for coalesced MN has been observed in several previous studies, and it highlights an unexpected benefit of co-encapsulating both materials that is not observed in nanohybrids containing single MN. SQUID magnetic measurements confirm that MHN retain the superparamagnetic characteristics of individual MN (see, e.g., FIG. 5). The MHN are thus detectable via both MRI and fluorescence at sub-micromolar Fe and sub-nanomolar QD concentrations (FIG. 2b), highlighting their utility for bimodal applications.

The disclosure also demonstrates imaging of the MHN structures of the disclosure in vitro. The ability of MHN to target and dual-mode image tumor cells was tested on MDA-MB-435 human cancer cells. To allow the MHNs of the disclosure to specifically target tumor cells, the MHNs were conjugated with the targeting ligand F3, a peptide known to target cell-surface nucleolin in endothelial cells in tumor blood vessels and in tumor cells and become internalized into these cells, and to transport a payload like nanoparticles or oligonucleotides into the tumor vasculature in vivo. Cells incubated with F3-conjugated MHN (F3-MHN) display dramatically increased NIR fluorescence and MRI contrast while cells incubated with unmodified MHN exhibit no significant fluorescence and MRI contrast (FIGS. 3a and 3b).

Simultaneous imaging and drug delivery was demonstrated using the anti-cancer drug DOX, which was incorporated into MHN during synthesis (˜0.093 mg of DOX per mg of MHN). The intrinsic fluorescence of DOX allows the independent imaging of DOX and QD contained in the MHN, which are observed to co-localize in some areas of MDA-MB-435 cells in vitro after 2 h of incubation (FIG. 3c). During a 24-h period, F3-MHN were observed to chaperone DOX into cancer cells and release it endosomally into the nuclei following tumor cell internalization (Inset in FIG. 3c). After 30 min of incubation with DOX-loaded F3-MHN (DOX-MHN-F3), the DOX fluorescence signal was mainly observed in the cytoplasm and co-localized with endosomes, whereas when free DOX was added, almost all of the DOX fluorescence signal was observed in the cell nuclei. As incubation time increases, the DOX in the cytoplasm was observed to translocate into the nuclei.

No significant toxicity of the MHN assemblies was observed, consistent with previous in vitro and in vivo studies with MQD and liposomal hybrid particles containing QD or MN. By contrast, DOX-incorporated F3-MHN display significant cytotoxicity which is higher than that of equivalent quantities of free DOX or DOX-incorporated untargeted MHN.

In addition to the in vitro cell assays, additional assays demonstrate the use of the MHNs in vivo. The utility of MHN was investigated for in vivo applications. MHN containing QD were synthesized that emit at NIR wavelengths (800 nm [MHN(800)]. This near infrared wavelength improves the imaging of organs by maximizing tissue penetration and minimizing optical absorption by physiologically abundant species such as hemoglobin. The PEGylated MHNs of the disclosure exhibit substantial blood circulation times (−3 h half-life), comparable to other PEG-nanomaterial formulations (˜0.5-2 h half-life for PEGylated carbon nanotubes and 0.2˜2.2 h for PEGylated QD). In addition, the MHNs survive circulation in the blood stream without dissociation into individual MN or QD as measured by TEM.

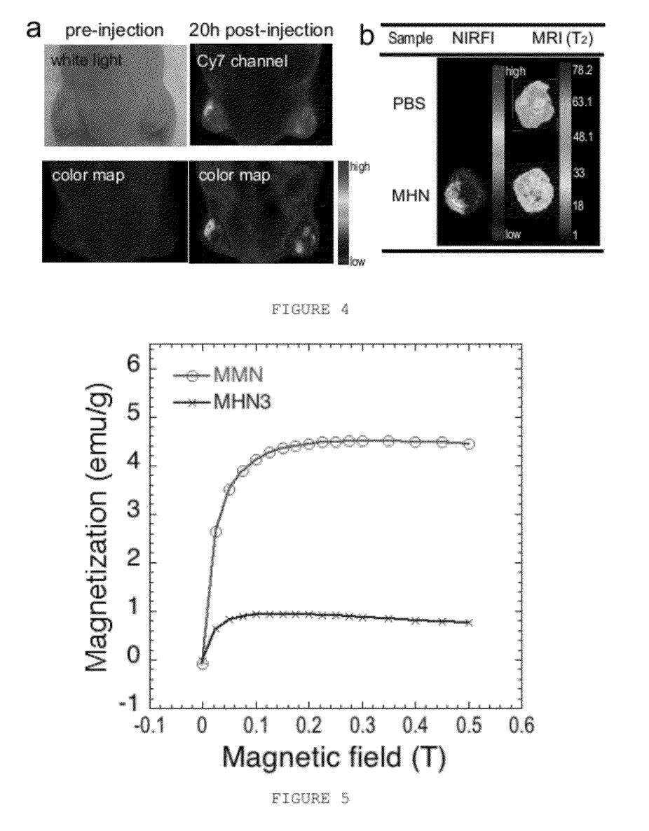

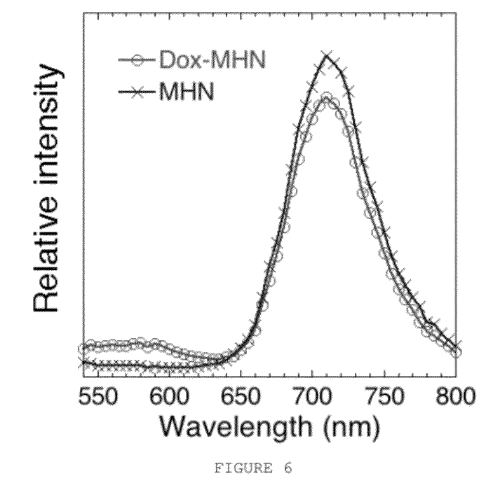

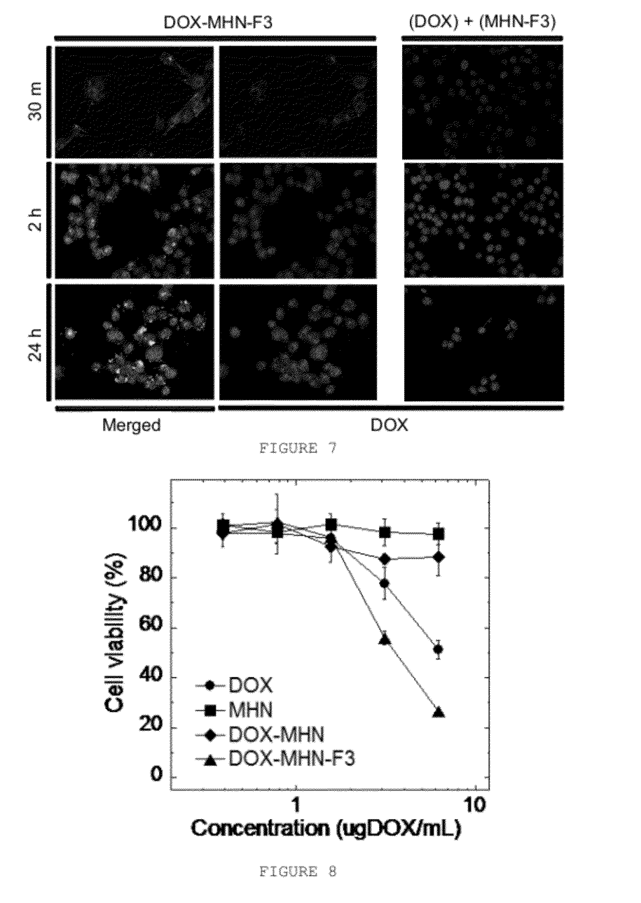

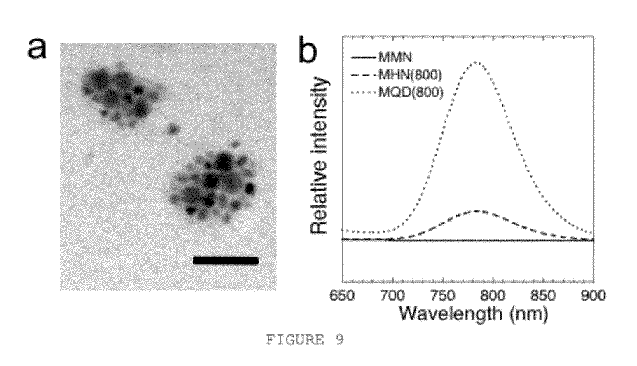

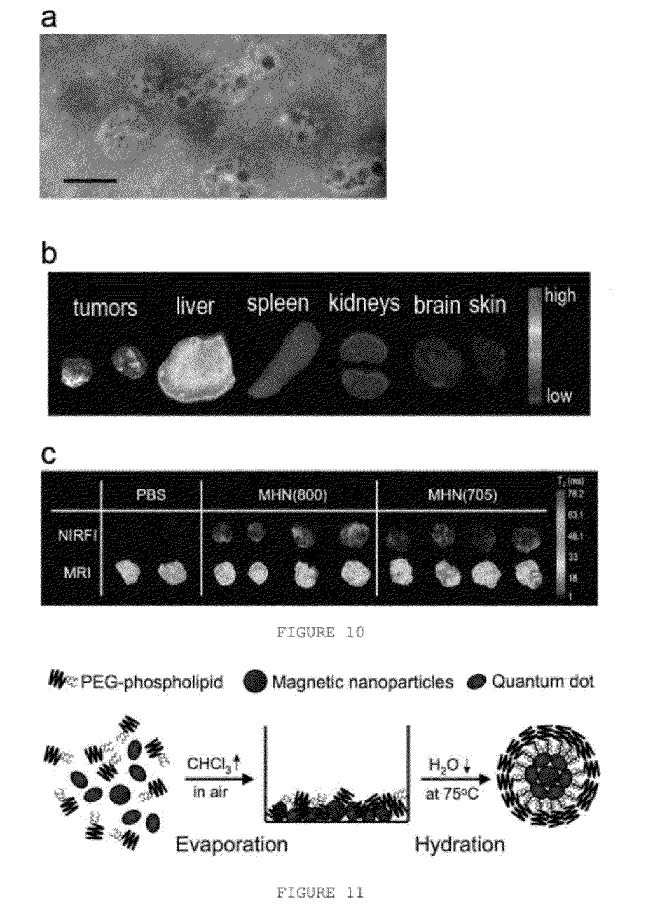

Long-circulating nanoparticles in the size range of 20-200 nm have been shown to accumulate preferentially at tumor sites through an enhanced permeability and retention effect. As an example MDA-MB-435 tumors-bearing nude mice were imaged prior to injection of MHNs and then 20 h after injection. In these optical images, significant fluorescence was observed in the tumors 20 h after MHN injection (FIG. 4a). Biodistribution measurements indicate that MHN mainly accumulate in the liver, while MHNs are not observed significantly in other organs (see FIG. 10b). To evaluate the multimodality of MHN in MR and optical imaging, the tumors were harvested 20 h after injection and immediately imaged in 4.7T MRI scanner and NIR optical imaging system. Significant differences in the optical and MRI contrast were observed between the tumors injected with PBS and those injected with MHN (FIG. 4b). The differences observed in the fluorescence images are much more substantial than in the MR images, due to the low background signals associated with NIR imaging. These examples demonstrate in vivo application of the MHNs of the disclosure due to the prolonged residence time in blood circulation displayed by MHN relative to other liposomal systems.

Accordingly, the disclosure provides compositions comprising a micelle hybrid nanostructures having bimodal imaging capacity and drug delivery capacity both in vivo and in vitro. Furthermore, the disclosure provides methods of making and using such micelle hybrid nanostructures. More particularly, the disclosure provides in specific embodiments, micellar hybrid nanostructure that comprise a magnetic nanostructure, at least one quantum dot, QD, and a therapeutic drug (e.g., an anti-cancer drug) within a single PEG-phospholipid micelle. The hydrophobic chains of the PEG-phospholipids interact strongly with hydrophobic character of the magnetic nanostructure (MN) and quantum dot material (QD), providing high dispersibility and stability for in vitro and in vivo applications. The MHN enable dual-mode imaging for cells in vitro and organs in vivo or ex vivo, combining the advantages of optical imaging (for microscopic resolution and in vivo fluorescent imaging) and MRI (for determination of full anatomical distribution in vivo). One of skill in the art will readily recognize that other magnetic nanostructures in addition to those specifically described herein can be used as well as other quantum dot materials. Accordingly, the methods and uses described herein are applicable to the synthesis of other hybrid nanodevices that combine the dissimilar functions of two or more nanomaterials such as MRI, photo-thermal therapy, Raman imaging, and optical imaging. Simultaneous dual-mode diagnosis and therapy with the hybrid system reported here may allow for more effective early detection and treatment of various types of cancers.

For example, an MHN can be used to deliver drugs such as doxyrubicin to a tissue followed by excitation of the nanoparticle to cause disruption of the micelle and delivery of the drug. Excitation can be accomplished by contacting the composition with an appropriate wavelength of light that causes excitation of the nanoparticle. The nanoparticle will have an increased vibration/thermal activity resulting in disruption of the micelle. This will result in delivery of drug only at the site where the excitation energy is delivered.

The following examples are intended to further described but not limit the disclosure.

### EXAMPLES

For the micellar hybrid nanoparticle (MHN) synthesis, 100 μL (MHN1), 300 μL (MHN3), or 500 μL (MHN5) of TOP-coated CdSe/ZnS or CdSeₓTe₁₋ₓ/ZnS quantum dots (QD, Invitrogen, CA, USA) in chloroform (2 mg/mL), and 100 μL of oleic acid-coated magnetic iron oxide nanoparticles (MN, prepared using a previously reported method of T. Hyeon, et al. J. Am. Chem. Soc. 2001, 123, 12798) in chloroform (2 mg/mL), were mixed with 200 uL of 1,2-dipalmitoyl-sn-glycero-3-phosphoethanolamine-N-[methoxy(polyethylene glycol)-2000] (PEG-PE or PEG-phospholipids, Avanti Polar Lipids, Inc., AL, USA) in chloroform (10 mg/mL). For the drug-incorporated MHN, 100 uL of doxorubicin (DOX, Sigma-Aldrich Chemicals, MO, USA) in chloroform containing triethylamine (TEA) (molar ratio 1:1=TEA:DOX, 1 mg/mL) was also added. After complete evaporation of the chloroform, the dried film was hydrated by adding 1 mL water at 75° C. and the synthesis vessel was placed in an ultrasonic bath for 5 min to obtain an optically clear suspension. The suspension was first filtered through a 0.1 μm membrane.

The MHN and micellar MN (MMN) were then selectively collected by trapping on a magnetic column (Miltenyi Biotec, Bergisch Gladbach, Germany) and rinsing with phosphate buffered saline (PBS) three times. Only the MHN and MMN were trapped on the magnetic column due to their magnetic properties, while micellar QD (MQD), DOX micelles, and empty micelles passed through the column. The MHN and MMN were eluted from the column after removal of the magnet, using 1 mL PBS. The eluted solution containing the MHN and MMN was then centrifuged at 14,000 rpm for 10 min and the supernatant, containing smaller individual MMN, was discarded. The MHN were re-suspended in deionized water or PBS solution.

The MMN or MQD shown in FIGS. 1e and 1f were prepared by encapsulating either hydrophobic MN or QD alone with PEG-phospholipids. For the MMN preparation, 100 μL of MN in chloroform (0.2 mg/mL) were mixed with 200 μL of PEG-phospholipids in chloroform (10 mg/mL). After complete evaporation of the chloroform, the dried film was hydrated by adding 2 mL of water at 75° C. and the synthesis vessel was placed in an ultrasonic bath for 5 min to obtain an optically clear suspension. The suspension was first filtered through a 0.1 μm membrane. The MMN were then selectively collected by trapping on the magnetic column and rinsing with PBS three times to remove empty micelles. For the MQD preparation, 100 μL of QD in chloroform (0.2 mg/mL) were mixed with 200 uL of PEG-phospholipids in chloroform (10 mg/mL). After complete evaporation of the chloroform, the dried film was hydrated by adding 2 mL water at 75° C. and the synthesis vessel was placed in an ultrasonic bath for 5 min to obtain an optically clear suspension. The suspension was first filtered through a 0.1 μm membrane. The MQD were then selectively collected by rinsing on a centrifuge filter (100,000 MWCO, Millipore) three times with PBS to remove empty micelles.

To determine the amount of DOX incorporated into the MHN, DOX-incorporated MHN were disrupted in 0.5 M HCl-50% ethanol overnight and the fluorescence intensity of DOX loaded in MHN was compared with a standard curve of DOX fluorescence in the same solution.

For transmission electron microscope (TEM) imaging, an aliquot of MMN, MQD, or MHN dispersed in water was dropped onto the carbon film covering a 300-mesh copper minigrid (Ted Pella, Inc., CA, USA), which was then gently wiped off after approximately 1 min and air-dried. For negative staining, the grid was incubated with pH 13 1.3% phosphotungstic acid for an additional 1 min. TEM images were obtained using a Hitachi H-600A transmission electron microscope. Hydrodynamic size of MMN, MQD or MHN was obtained using a Zetasizer ZS90 dynamic light scattering machine (Malvern Instruments, Worcestershire, UK).

The photoluminescence (PL) spectra of MMN, MQD or MHN were obtained using a 450 nm excitation source with an Acton 0.275-m monochromator, 480-nm cutoff filter, and a UV-enhanced liquid nitrogen-cooled, charge-coupled device (CCD) detector (Princeton Instruments, NJ, USA). The collection optics consisted of a 2.54 cm diameter microscope objective lens coupled to fiber-optic cable.

For NIR fluorescence imaging and MRI T2 mapping, MMN, MQD, or MHN serially diluted in PBS, and cells incubated with/without MHN for 2 h were placed in a 386-well plate, containing 95 μl total sample/well. The tumors injected with PBS or MHN were placed in flat plastic plate. The optical images were obtained in the Cy5.5 channel (excitation at 680 nm/emission at 720 nm) or the Cy7 channel (excitation at 760 nm/emission at 800 nm) with a NIR fluorescence scanner (LI-COR biosciences, NE, USA). The MRI was performed using a 7 cm bore, Bruker (Karlsruhe, Germany) 4.7 T magnet. R₂ is longitudinal relaxation rate equal to the reciprocal of the T₂ relaxation time (R₂=1/T₂) and it is calculated with a T₂-weighted MRI map. The fluorescence and MR images were analyzed using the OsiriX program (Apple). For magnetic measurement, freeze-dried MMN or MHN were placed in gelatin capsules and the capsules were inserted into transparent plastic drinking straws. The measurements were performed at 298 K using a MPMS2 superconducting quantum interference device (SQUID) magnetometer (Quantum Design, CA, USA). The samples were exposed to direct current magnetic fields in stepwise increments up to 0.5 Tesla. Corrections were made for the diamagnetic contribution of the capsule and straw. The magnetic data were used to quantify the amount of MN in the MHN.

(SEQ ID NO: 1)
KDEPQRRSARLSAKPAPPKPEPKPKKAPAKK (F3)

peptide has been shown to bind preferentially to blood vessels and tumor cells in various tumors. The peptide was synthesized using Fmoc chemistry in a solid-phase synthesizer, and purified by preparative HPLC. The sequence and composition of the peptide was confirmed by mass spectrometry. For further conjugation, an extra cysteine residue was added to the N-terminus. For conjugation with F3, 5% of the PEG-PE used in the MHN synthesis was replaced with 1,2-Distearoyl-sn-Glycero-3-Phosphoethanolamine-N-[Maleimide (Polyethylene Glycol)2000] (maleimide PEG-PE, Avanti Polar Lipids, AL, USA). 200 μg of F3 was reacted with 2 mg maleimide-activated MHN in PBS. After incubation for 30 min at room temperature, the F3-modified MHN sample was purified on a desalting column (Pall, N.Y., USA).

For in vitro studies, MDA-MB-435 human carcinoma cells were maintained in Dulbecco's Modified Eagle's Medium (DMEM) supplemented with 10% fetal bovine serum (FBS) and 100 μg/ml penicillin-streptomycin. For fluorescence microscopy, the cells (3000 cells per well) were seeded into 8-well chamber slides (Lab-Tek) overnight. The cells were then incubated with 50 μg of MHN or F3-MHN per well for 2 h (for intracellular targeting) and 50 μg of DOX-loaded F3-MHN (0.093 mg DOX per mg MHN) or 4.5 μg of free DOX (equivalent with DOX amount for DOX-loaded F3-MHN) physically mixed with 50 μg of F3-MHN per well for 30 min, 2 h, and 24 h (for intracellular drug delivery) at 37° C. in the presence of 10% FBS. For the intracellular targeting study, the cells were observed without any fixation using an inverted fluorescence microscope (Nikon, Tokyo, Japan). For the intracellular drug delivery study, the cells were fixed with 4% paraformaldehyde for 20 min, mounted in Vectashield Mounting Medium with 4′,6-diamidino-2-phenylindole (DAPI; Vector Laboratories, Burlingame, Calif.), and observed with a fluorescence microscope. For endosome staining, the cells incubated with DOX-incorporated F3-MHN for 30 min were fixed with 4% paraformaldehyde for 20 min, and permeabilized and blocked with the solution containing 1% bovine serum albumin (BSA) and 0.1% Triton X-100 in PBS for 30 min, incubated with 2 μg/mL endosome marker (EEA1 antibody, Abcam) for 1 h, and then with 2 μg/mL AlexaFluor® 488 conjugated goat anti-mouse IgG antibody for 1 h at room temperature. The nuclei stained with DAPI were observed in blue channel (excitation at 360 nm/emission at 460 nm). The DOX fluorescences were observed in Cy3 channel (excitation at 540 nm/emission at 580 nm). The QD fluorescence of MHN(705) were observed in Cy5.5 channel.

For cytotoxicity test, MDA-MB-435 human carcinoma cells were incubated with free DOX, MHN, DOX-loaded MHN, and DOX-loaded F3-MHN with different concentrations (equivalent amount of DOX and MHN, n=3) for 4 h, rinsed with cell medium three times, and then incubated for an additional 44 h. The cytotoxicity of various formulations of MHN was evaluated using MTT assay (Invitrogen). Cell viability was expressed as the percentage of viable cells compared with controls (cells treated with PBS).

To quantify blood half-life, MHN(800) in PBS (100 μL) were intravenously injected into nude BALB/c mice (n=3) at a dose of 3 mg/kg. Heparinized capillary tubes (Fisher) were used to draw 15 μL of blood from the periorbital plexus at different times after intravenous injection. The extracted blood samples were immediately mixed with 10 mM EDTA (in PBS) to prevent coagulation. The blood extracted at different times was imaged in a 96-well plate in Cy7 channel using the NIR fluorescence scanner and the blood half-life was calculated by fitting the fluorescence data to a single-exponential equation using a one-compartment open pharmacokinetic model [4].

To determine if the MHN are dissociated during in vivo circulation, ˜0.5 mL blood was extracted from the mouse 1 h after intravenous injection of MHN (10 mg/kg) and immediately mixed with ˜0.5 mL of 10 mM EDTA (in PBS) to prevent coagulation. The MHN were recovered from the blood mixture by rinsing on the magnetic column 5 times with PBS. Their size and shape were observed using TEM with negative staining by pH 13 1.3% phosphotungstic acid (Note that the TEM used here can detect the micelle coating layer as well as MN and QD).

To quantify in vivo tumor accumulation, MDA-MB-435 human carcinoma tumors were subcutaneously implanted bilaterally into the hind flanks of nude BALB/c mice. Tumors were used when they reached ˜0.5 cm in size. All animal work was reviewed and approved by Burnham Institute for Medical Research's Animal Research Committee. The MHN (or PBS control) samples were intravenously injected into mice (n=2-4) with a dose of 10 mg/kg. For real-time observation of tumor uptake, mice were imaged under anesthesia in Cy7 channel using the NIR fluorescence scanner, both pre- and 20 h post-injection of MHN(800). To determine biodistibution, mice were sacrificed 20 h after MHN(800) injection by cardiac perfusion with PBS under anesthesia, and the organs were dissected and imaged in Cy5.5 or Cy7 channel using the NIR fluorescence scanner.

The invention has been generally described. One of skill in the art will recognize that variations can be made without departing from the spirit and scope of the following claims.

1. A micelle compositions encapsulating a plurality of different nanostructures at least two of the plurality of nanostructures having different excitation/emission spectrums or detectable signals.

2. A micelle compositions of claim 1, wherein at least two of the plurality of nanostructure comprising different materials.

3. The composition of claim 2, wherein at least one nanostructure comprises a magnetic material.

4. The composition of claim 1, wherein the at least one nanostructure comprises a quantum dot.

5. The composition of claim 1, wherein the plurality of nanostructures comprise at least one quantum dot and at least one magnetic nanostructure.

6. The composition of claim 1, wherein the composition further comprises a therapeutic drug.

7. The composition of claim 6, wherein the therapeutic drug is an anticancer drug.

8. The composition of claim 7, wherein the anticancer drug is selected from the group consisting of methotrexate, fluorouracil, hydroxyurea, mercaptopurine, cisplatin, daunorubicin doxorubicin, etoposide, Vinblastine, Vincristine and Pacitaxel.

9. The composition of claim 1, further comprising a targeting moiety linked to the micellar structure.

10. The composition of claim 1, wherein a micelle lipid is pegylated.

11. A method of making a pegylated-micelle-nanostructure composition comprising:
evaporating a mixture comprising pegylated lipids, at least one nanostructure, at least one quantum dot and an organic solvent to obtain a dry mixture;
hydrating the dry mixture in a hydrating medium to obtain a pegylated-micelle-nanostructure composition, wherein the nanostructure and quantum dot are encapsulated within the micelle.

12. The method of claim 11, further comprising adding a drug to either of the organic solvent or the hydrating medium.

13. The method of claim 11, wherein the pegylated lipid comprises a targeting moiety.

14. The method of claim 12, wherein the drug is an anti-cancer agent.

15. The method of claim 14, wherein the anticancer agent is selected from the group consisting of methotrexate, fluorouracil, hydroxyurea, mercaptopurine, cisplatin, daunorubicin doxorubicin, etoposide, Vinblastine, Vincristine and Pacitaxel.

16. A composition made by the method of claim 11.

17. A pharmaceutical composition comprising a micelle containing a plurality of nanostructures of claim 16 and a pharmaceutically acceptable carrier.

18. A method of treating or diagnosing a disease or disorder in a subject comprising administering the composition of claim 17 to a subject and contacting the subject with a device that can detect the magnetic rotation of a nanostructure.

19. A method of treating or diagnosing a disease or disorder in a subject comprising administering the composition of claim 17 to a subject and contacting the subject with a device that excites the nanostructure to induce vibration or thermal energy and the site of the nanostructure.
